(19) United States
(12) Patent Application Publication
(10) Pub. No.: US 2021/0346612 A1
(43) Pub. Date: Nov. 11, 2021

(54) MESH NEBULISERS

(71) Applicant: BANKSIDE INTERNATIONAL LTD., Devizes (GB)

(72) Inventors: Kyoungwoo Kim Blick, London (GB); Stephen Anthony Sayers, Weston Turville (GB)

(21) Appl. No.: 17/220,852

(22) Filed: Apr. 1, 2021

Related U.S. Application Data
(63) Continuation-in-part of application No. 17/276,096.

(30) Foreign Application Priority Data
Sep. 19, 2018 (GB) 1815253.8

(51) Int. Cl. A61M 11/00

(52) U.S. Cl. CPC A61M 11/003; A61M 15/0085

(57) ABSTRACT

A mesh for use in a mesh nebuliser. The mesh 300 comprises a first plurality of apertures 310 extending through the mesh 300, the first plurality of apertures 310 having a first average diameter and a second plurality of apertures 320 extending through the mesh 300, the second plurality of apertures 320 having a second average diameter. The first average diameter is different to the second average diameter. The first average diameter may be sized to produce droplet of 1-5 μm, whereas the second average diameter may be sized to produce droplets of 5-30 μm. There is also provided a mesh nebuliser having the present mesh.

### RELATED APPLICATIONS

The present application is a continuation-in-part of U.S. patent application Ser. No. 17/276,096, filed Mar. 12, 2021, which is a 371 Nationalization of PCT/EP2019/075057 filed Sep. 19, 2019, which claims the benefit of United Kingdom Patent Application No. 1815253.8 filed Sep. 19, 2018, the entire specifications of which are hereby incorporated by reference in their entirety for all purposes.

This invention is a mesh for use in a mesh nebuliser, and separately to a nebuliser having a mesh.

Mesh nebulisers are an increasingly popular choice of nebuliser for providing a dosage of medication to a patient. Mesh nebulisers generally comprise a body containing, or in fluid communication with, a reservoir of liquid comprising a medicament to be nebulised.

There are two main types of mesh nebulisers: passive and active. Active mesh nebulisers, further comprises a mesh which is generally surrounded by a piezoelectric or similar, the piezoelectric having a power source that selectively supplies power to the piezoelectric. The mesh contains, on average, 500 to 4,000 apertures. Meshes for use in active mesh nebulisers are generally domed in shape, and sit within a ring of piezoelectric.

The liquid in the reservoir is directable to the mesh. When actuated by the patient, the power source causes the piezoelectric, and thus the mesh, to vibrate and the vibration of the mesh causes the liquid to be turned into an aerosol of liquid droplets which are then inhaled by the user.

Passive, also referred to as static, mesh vibrators on the other hand are arranged so that the piezoelectric element is mounted in close proximity to, but not in contact with, a fixed mesh, which is generally planar. The mesh contains, on average, 500 to 4,000 apertures. The liquid to be nebulised is fed in between the mesh and the piezoelectric. When actuated, the piezoelectric vibrates and forces the liquid through the mesh.

For medical use, the droplets produced by either type of mesh nebuliser generally have a mass medium aerodynamic (MMAD) in the range of 1-5 μm. Droplets of this size are able to pass into the alveoli of a patient's lung and are thus particularly bioavailable. Mesh nebulisers are advantageous over other forms of nebuliser because they are able to provide a narrow distribution of particle sizes at low velocity to ensure that as much medication as possible is bioavailable. Furthermore, as no active ingredient will impact the mouth or throat, there will be little or no taste sensation or throat irritation.

The present invention, on the other hand, finds utility with mesh nebulisers for recreational purposes. Non-medical uses of nebulisers include the pastime of “vaping” where a liquid containing an active ingredient such as nicotine in a water or oil-based carrier are inhaled. Furthermore, the liquid might contain a flavour so that the user not only gets the effects of the active ingredient but also the flavour. Prior art vaping devices will produce a large range of particle sizes which will contain not only small particles in the range of 1-5 μm that will be absorbed by the alveoli but also particles of over 10 μm that will deposit in the mouth and back of the throat to produce a taste sensation. Particles will also be in the order of 5-10 μm and will pass into the trachea where they will generally neither be bioavailable nor produce a taste sensation. Particles of these sizes will however create a so-called “throat hit” which may contribute to the user's experience. Furthermore, droplets less than 1 μm will pass into the lungs but then be exhaled without being absorbed. Such exhalation will create a cloud of vapour which some users consider part of the vaping experience. Furthermore, vaping devices rely on heat to produce vapour/aerosols and the addition of heat often leads to the production of many undesirable toxicants.

Mesh nebulisers intended for medical use do not use heat and therefore have a very low toxicant profile, but have not been considered for recreational uses since the band of particle sizes created is too narrow. Therefore, either a bioavailable particle size could be created or a taste sensation but not both, thus the user experience is less than would be achieved from current smoking, heating or vaping devices. For this reason, mesh nebulisers are not used as recreational vaping devices.

Two main methods are currently used for mesh production. These are electroplating and laser cutting techniques, both of which produce a tapered or conical aperture. A conical aperture amplifies flow at the nozzle and reduces viscose losses, and is therefore desired. The electroplating method employs a lithographic plate and the duration of the electroplating process is varied to create different aperture sizes. Laser cutting involves the use of a laser beam to cut the mesh holes into a thin sheet of metal or polymer material.

Most mesh nebulizer meshes are constructed from a metal alloy, which can include platinum, palladium, nickel and stainless steel.

The present application seeks to address this problem and provide a mesh for use in a mesh nebuliser that could be used for recreational purposes, or where a flavour is required in a medical setting to encourage patient compliance, such as if used by young children. Therefore, according to an aspect of the present invention, there is provided a mesh for use in a mesh nebuliser, the mesh comprising a first plurality of apertures extending through the mesh, the first plurality of apertures having a first average diameter; and at least one second aperture extending through the mesh, the at least one second aperture having a second average diameter, wherein the first average diameter is different to the second average diameter.

Preferably, the first plurality of apertures has an average diameter in the range of 0.5-2.5 μm and the at least one second aperture has a diameter in the range of 2.5-15 μm. Aperture size may be measured at the smallest point of the aperture—apertures in nebuliser meshes will generally be conical in shape—using any technique known in the art, such as pixel count. Apertures having an average diameter of 0.5-2.5 μm will produce droplets having an average diameter in the range of 1-5 μm and apertures having an average diameter in the range of 2.5-15 μm will produce droplets having an average diameter in the range of 5-30 μm.

Where referred to herein, the term “diameter” means an average diameter. The apertures may be of any shape including, but not limited to, circular, oval, square, rectangular, pentagonal, hexagonal and be regular or irregular.

The first plurality of apertures are preferably sized so as to produce particle sizes with an MMAD in the range of 1-5 μm. Therefore, the first plurality of apertures will produce particle sizes that will be absorbed by the alveoli of a user's lungs and thus make the nebulised liquid bioavailable to the user. MMAD may be determined by any known method such as by cascade impactor.

The at least one second aperture are preferably sized so as to produce a particle size with an MMAD in the range of 5-30 μm, and preferably an MMAD in the range of 10-30 μm. Such particles will impact in the nasopharynx and thus produce a taste sensation for the user. Therefore, a mesh with apertures in the range of 1-5 μm and 10-30 μm will produce droplets sized so that a user will receive the benefit of a bioavailable substance and also a flavour component.

The first plurality of apertures may be arranged on one side of the mesh and the at least one second aperture may be arranged on another side of the mesh.

Alternatively, the first plurality of apertures may be arranged at the centre of the mesh and the at least one second aperture may be arranged on the outside of the first plurality of apertures.

Alternatively, the at least one second aperture may be arranged at the centre of the mesh and the first plurality of apertures may be arranged on the outside of the at least one second aperture.

Alternatively, the first plurality of apertures and the at least one second aperture may be distributed evenly on the mesh.

Alternatively, where there is provided a plurality of the at least one second aperture, the first plurality of apertures and the plurality of second apertures may be arranged as alternate lines or rows across the width of the mesh or one of the first or second plurality of apertures may be arranged in a cross pattern or be arranged to extend radially from the centre of the mesh, with the other of the first or second plurality of apertures arranged therebetween.

The choice of arrangement of first or second apertures will depend on various factors, including and not limited to different manufacturing methods (for example, meshes produced by laser or electrolysis) which will suit some configurations. Some configurations of aperture will provide a far more even spread of different particle sizes. Some configurations of aperture will be less prone to blockages, especially with regards to the smaller diameters. Some configurations will work better in active or passive meshes. For example, with meshes used in active nebulisers the mesh will deform and so a different configuration may be preferred.

Preferably, the first plurality of apertures comprises a range of 0.1%-99.9% of the apertures by number and the at least one second aperture comprises a range of 0.1%-99.9% of the apertures by number, wherein the total proportion of both sizes of aperture amounts to 100%. Alternatively, the first plurality of apertures comprises a range of 0.1%-10% of the apertures by number and the at least one second aperture comprises a range of 90%-99.9% of the apertures by number. Alternatively, the first plurality of apertures comprises a range of 11%-20% of the apertures by number and the at least one second aperture comprises a range of 80%-89% of the apertures by number. Alternatively, the first plurality of apertures comprises a range of 21%-30% of the apertures by number and the at least one second aperture comprises a range of 70%-79% of the apertures by number. Alternatively, the first plurality of apertures comprises a range of 31%-40% of the apertures by number and the at least one second aperture comprises a range of 60%-69% of the apertures by number. Alternatively, the first plurality of apertures comprises a range of 41%-50% of the apertures by number and the at least one second aperture comprises a range of 50%-59% of the apertures by number. Alternatively, the first plurality of apertures comprises a range of 51%-60% of the apertures by number and the at least one second aperture comprises a range of 40%-49% of the apertures by number. Alternatively, the first plurality of apertures comprises a range of 61%-70% of the apertures by number and the at least one second aperture comprises a range of 30%-39% of the apertures by number. Alternatively, the first plurality of apertures comprises a range of 71%-80% of the apertures by number and the at least one second aperture comprises a range of 20%-29% of the apertures by number. Alternatively, the first plurality of apertures comprises a range of 81%-90% of the apertures by number and the at least one second aperture comprises a range of 10%-19% of the apertures by number. Alternatively, the first plurality of apertures comprises a range of 91%-99.9% of the apertures by number and the at least one second aperture comprises a range of 0.1%-9% of the apertures by number.

The at least one second aperture may comprise a single aperture, which may be sufficiently sized to produce a droplet that has an effect on the user experience. For example, a single second aperture could produce a relatively large droplet of over 10 μm that will impact the mouth and produce a taste. Alternatively, there may be a plurality of second apertures.

The selection of the range of the amount of the first and second aperture sizes will depend on the composition being nebulised. If the composition, for example, contained an aqueous solution comprising nicotine and a flavouring, the percentage of each aperture size will depend on the relative concentrations of the nicotine and the flavouring in order to get the desired user effect. For example, there may be provided a mesh having a range of 10-19% by number of apertures for the first plurality of apertures, which may have an aperture size producing droplets of 1-5 μm and 81-90% by number of apertures and the at least one second aperture, which may have an aperture size producing droplets of 10-30 μm. This arrangement may make bioavailable enough nebulised nicotine to the user's alveoli, and also provide enough nebulised flavouring to the user's mouth and throat to provide a desired user experience. Different concentrations of composition may require different percentages of different aperture sizes.

The mesh may comprise a third plurality of apertures having a diameter different from the average diameter of the first and second apertures. A third plurality of apertures would produce droplets of a different size, and so could provide a further user experience. The third plurality of apertures may comprise a diameter of less than 0.5 μm and preferably in the range of 0.05-0.5 μm, or alternatively 0.1-0.4 μm.

The third plurality of apertures may be sized so as to produce droplet sizes with an MMAD in the range of 0.1-1 μm, or alternatively 0.2-0.8 μm. Sub-micron diameters of particles will be so small as to pass into the lungs and then to be exhaled by the user without being absorbed. Such particle sizes will be of no use in a medical context but for recreational vapers, the exhaled particles will create a cloud of vapour which can be seen as part of the user's experience.

In particular, the exhaled vapour may provide the user with the experience of smoking a cigarette, rather than using an item of medical kit. This might be important for the user when trying to give up smoking cigarettes. Furthermore, if the liquid being nebulised is scented for flavour, then the resultant exhaled vapour will also be scented, which can may also be important for the user experience.

The third plurality of apertures may alternatively have a diameter in the range of 2.5-5 μm.

The third plurality of apertures may alternatively be sized so as to produce droplet sizes with an MMAD in the range of 5-10 μm. Diameters of particles in this size will impact into the larynx and produce a throat hit. Such particle sizes will be of no use in a medical context but for recreational vapers, the throat hit may produce a sensation akin to smoking cigarettes and therefore be appealing to some users, particularly those hoping to give up smoking cigarettes and who want to recreate the experience of cigarette smoking.

The first plurality of apertures may be arranged on one side of the mesh and the at least one second aperture may be arranged on another side of the mesh, with the third plurality of apertures arranged in between. Alternatively, third plurality of apertures may be arranged on one side of the mesh and the at least one second aperture may be arranged on another side of the mesh, with the first plurality of apertures arranged in between. Alternatively, the first plurality of apertures may be arranged on one side of the mesh and the third plurality of apertures may be arranged on another side of the mesh, with the at least one second aperture arranged in between

Alternatively, the first plurality of apertures may be arranged at the centre of the mesh and the at least one second aperture may be arranged on the outside of the mesh, with the third plurality of apertures in between. Alternatively, the at least one second aperture may be arranged at the centre of the mesh and the first plurality of apertures may be arranged on the outside of the mesh with the third plurality of apertures in between. Alternatively, the third plurality of apertures may be arranged at the centre of the mesh and the at least one second aperture may be arranged on the outside of the mesh, with the first plurality of apertures in between. Alternatively, the third plurality of apertures may be arranged at the centre of the mesh and the first plurality of apertures may be arranged on the outside of the mesh with the at least one second aperture in between. Alternatively, the first plurality of apertures may be arranged at the centre of the mesh and the third plurality of apertures may be arranged on the outside of the mesh, with the at least one second aperture in between. Alternatively, the at least one second aperture may be arranged at the centre of the mesh and the third plurality of apertures may be arranged on the outside of the mesh with the first plurality of apertures in between.

The first and/or second and/or third plurality of apertures could extend radially from the centre of the mesh, or be arranged in alternating rows.

The first plurality of apertures, the at least one second aperture and the third plurality of apertures may be distributed evenly on the mesh.

The first plurality of apertures could comprise 0.1-97% of the total apertures by number, the at least one second aperture could comprise 0.1-97% of the total apertures by number and the third plurality of apertures could comprise 0.1-97% of the total apertures by number, with the total percentage of each plurality of apertures adding up to 100%. The ratio of first plurality of apertures to at least one second aperture to third plurality of apertures could be 33%:33%:33% by number of apertures.

Further still, there could be provided a mesh comprising a fourth plurality of apertures having a diameter different from the first, second and third apertures. The fourth plurality of apertures may have an average diameter of less than 0.5 μm and preferably in the range of 0.05-0.5 μm, or an average diameter of in the range of 2.5-5 μm.

The fourth plurality of apertures may be sized so as to produce droplet sizes with an MMAD in the range of 0.1-1 μm or 5-10 μm. A mesh with four sizes of aperture could produce droplet sizes in the ranges of less than 0.1 μm up to 30 μm in average diameter, for the full range of user experience not available from prior art mesh nebulisers.

A mesh with four aperture sizes may be arranged in any suitable manner, such as described above in relation to meshes with two or three different average sizes of aperture.

According to a second aspect of the invention, there is provided a mesh for a mesh nebuliser, the mesh comprising at least one aperture shaped so that, in use, nebulised droplets ejected from the aperture comprise at least two average diameters.

Meshes according to the second aspect of this invention could comprise at least one aperture having regions of different average diameters conjoined by relatively narrow regions. When nebulised, liquid will pass through the regions of different average diameters, but not through the relatively narrow region.

The at least one aperture may comprise two, three, four or more regions of different average size conjoined by relatively narrow regions.

For example, the at least one apertures could comprise an irregular hourglass shape, comprising a first part and a second region conjoined with a narrow region, the first and second regions having different average diameters to produce different sizes droplets. The first and second regions could have average diameters as described in relation to first plurality of apertures and the at least one second aperture of the first aspect of the present invention.

According to a third aspect of the invention, there is provided a mesh nebuliser comprising a mesh as previously described. The mesh nebuliser may be an active or a passive mesh nebuliser. The mesh in the mesh nebuliser may be interchangeable, so that the user may replace or clean the mesh, or so that they user may be able to alternate between different user experiences based on different mesh aperture sizes. Different users will also have different general preferences. Also these may change over time. For example, for a smoker or vaper, often throat hit, taste and exhalation are important effects. However, over time, the need for some of the effects diminishes (as has been shown in vaping studies). As the need diminishes, it gives the user the opportunity to reduce some of the effects and focus on those that are important to them. Additionally, users may wish to switch meshes at different times of the day or in different circumstances. For instance in a public space or possibly at home, the user may wish to use a mesh where there is little or no exhalation. Also for formulas being nebulised where the user is merely after the therapeutic effect of an active ingredient in the formula, some users may want a simpler experience where nebulised droplets are directed to their alveoli and nowhere else, whereas others may prefer an element of feedback via taste or throat hit sensation. A range of meshes gives users an option to semi-personalise the experience, which will improve the experience for a consumer of nicotine or other ingredient or increase adherence for a medical or wellness use.

For example, a user may chose a mesh which produces no droplets in the range of 0.1-1 μm so that there is no exhalant, or one which produces no droplets in the range of 5-10 μm so as to produce no throat hit.

The mesh nebuliser may comprise a mesh, and a piezoelectric element in communication with a power source. In the case of an active mesh nebuliser, the piezoelectric element may be arranged about the mesh, and in the case of a passive mesh nebuliser the piezoelectric may be arranged apart from the mesh.

The mesh nebuliser may comprise a mouthpiece from which a user draws nebulised vapour. The mesh nebuliser may comprise a reservoir to contain a liquid to be nebulised. The reservoir may comprise replaceable cartridges.

The mesh nebuliser may be actuatable with a switch. The switch may be manually activated by the user, such as with a push button. Alternatively, the mesh switch may be activated by way of a microphone which senses a change in sound when a user draws from the nebuliser.

Embodiments of the present invention will be described by way of example only with reference to the following drawings.

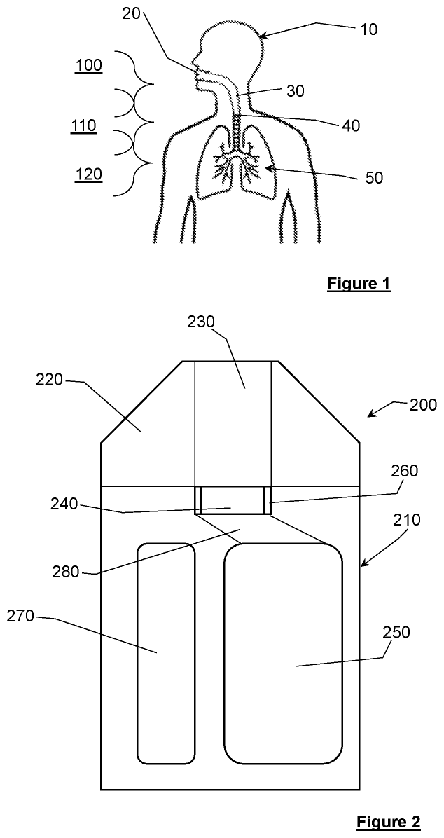

FIG. 1 shows a schematic of the human respiratory system showing where droplets of different diameters impact during inhalation of nebulised fluids;

FIG. 2 shows a schematic diagram of a mesh nebuliser;

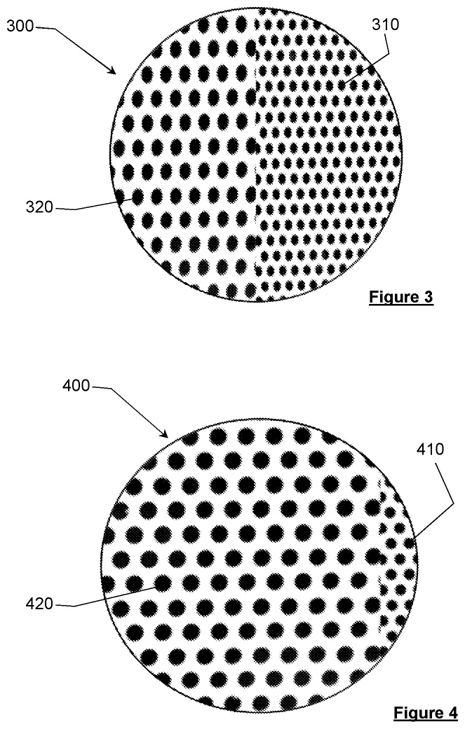

FIG. 3 shows a schematic mesh with apertures of two different sizes;

FIG. 4 shows an alternative mesh with apertures of two different sizes;

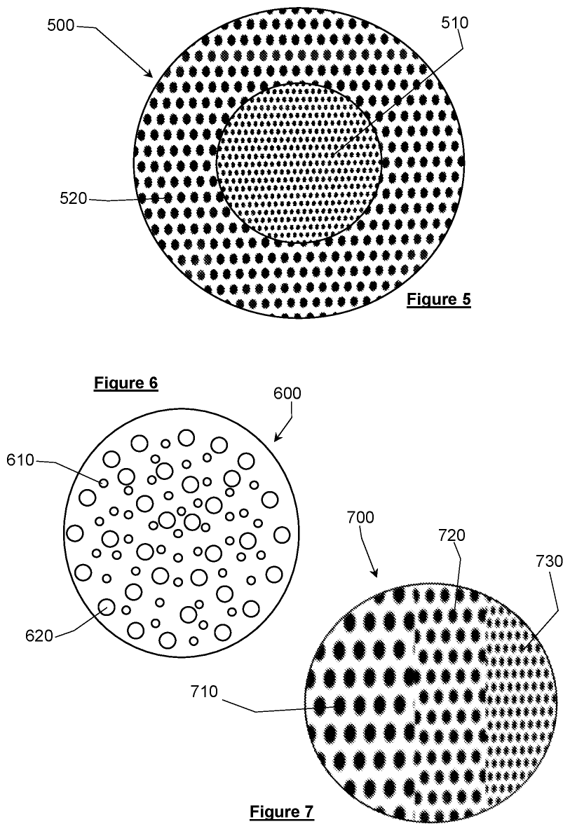

FIG. 5 shows an alternative mesh with apertures of two different sizes;

FIG. 6 shows an alternative mesh with apertures of two different sizes;

FIG. 7 shows an alternative mesh with apertures of three different sizes;

FIG. 8 shows an alternative aperture;

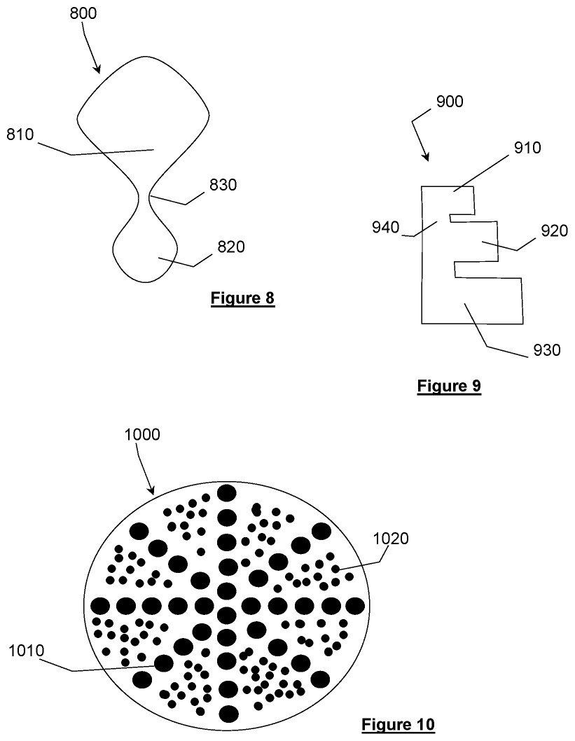

FIG. 9 shows an alternative aperture; and

FIG. 10 shows an alternative mesh with apertures of two different sizes.

Turning first to FIG. 1, there is shown a schematic diagram of the human respiratory system showing in outline a person generally indicated 10 having a mouth 20 throat 30, trachea 40 and lungs generally indicated 50. The lungs comprise bronchioles and alveoli. As shown by bracket 100, inhaled aerosol droplets having diameters of over 10 μm will deposit in the mouth 20 and throat 30. As indicated by bracket 110, inhaled aerosol droplets having diameters of 5 to 10 μm will deposit on the trachea, and as indicated by bracket 120, droplets having a particle size of 1 to 5 μm will be deposited in the alveoli of the lungs. Particles of less than 1 μm will generally be exhaled and will generally not be deposited or absorbed into the respiratory system, other than through agglomeration.

Turning now to FIG. 2, there is shown a schematic of a mesh nebuliser, generally indicated 200. In this example, the mesh nebuliser is a vibratable mesh nebuliser, also referred to as an active nebuliser. The mesh nebuliser comprises a body generally indicated 210 comprising a mouthpiece 220 having a central conduit 230 in communication with a mesh 240. The body 210 contains a reservoir 250 containing a liquid to be nebulised. The mesh 240 is surrounded by a piezoelectric element 260 which is connected to a power source such as a battery 270. A further conduit 280 connects the reservoir 250 to the mesh 240.

In use a user will inhale on the mouthpiece 220 to draw liquid from the reservoir 250 through the further conduit 280 to the mesh 240. The user may actuate the piezoelectric element 260 such as by pressing a switch to provide power from the power source 270 to the piezoelectric element 260. The piezoelectric element 260 vibrates the mesh 240, which causes the liquid to pass through apertures in the mesh 240 and be turned into aerosol droplets. The aerosol droplets are drawn through the conduit 230 in mouthpiece 220 to the user.

Turning now to FIG. 3, there is shown an embodiment of mesh according to the present invention, which will be used in a mesh nebuliser. There is shown a mesh generally indicated 300 comprising a first plurality of apertures generally indicated 310 and a second plurality of apertures generally indicated 320. The first plurality of apertures 310 are smaller than the apertures 320. The apertures shown in FIG. 3 are not drawn to scale and are schematic for illustrative purposes. Whilst there may be a total of between 1000 to 4000 apertures on a prior art mesh having a single average diameter size, depending on the choice of aperture sizes, meshes according to the present invention may comprise between 50 and 5000 apertures. The first plurality of apertures may be 50% of the total apertures by number or by area. Mesh 300 is generally circular with opposed planar surfaces, but equally can be of irregular size and shape.

As shown in FIG. 3, one side of the mesh comprises the first plurality of apertures 310 and the other side of the mesh comprises the second plurality of apertures 320.

Turning to FIG. 4, there is shown a mesh, generally indicated 400, comprising a first plurality of apertures 410 and a second plurality of apertures 420. The first plurality of apertures 410 are smaller than the apertures 420 Compared to the mesh of FIG. 3 there are a larger number of the second plurality of apertures 420 than there are of the first plurality of apertures 410. In such an embodiment, it may be that the number of apertures of each size may be selected depending on the user experience required. For example, turning to FIG. 2, the liquid to be nebulised in the reservoir 250 may comprise two different elements. For example, the liquid may comprise nicotine which is absorbed into the body through the alveoli of the lungs and a flavour component such as menthol, which will only be tasted if it is deposited onto the mouth of the user. Therefore, the first plurality of apertures 310, 410 may be sized so as to produce droplet sizes of 1 to 5 μm so that a liquid containing nicotine will be nebulised by the mesh and the nicotine will be bioavailable to the user since the droplets are the right size to enter the alveoli where the nicotine will be absorbed.

Furthermore, the second plurality of apertures 320, 420 may be sized so as to produce nebulised droplets of over 10 μm in diameter, so that those droplets will be deposited in the users mouth. The user will be able to taste the flavour. The respective proportion of first and second pluralities of apertures will be varied depending on the desired user experience and the respective concentrations of the liquid to be nebulised.

FIG. 5 shows an alternative arrangement of apertures. There is shown a mesh generally indicated 500 comprising a first plurality of apertures 510 arranged towards the centre of the mesh 500, and a second plurality of apertures generally indicated 520 arranged on the outside of the mesh 500. As described with respect to FIGS. 3 and 4, the relative areas of the first and second plurality of apertures, and therefore the number of apertures, can be varied depending on the desired user experience.

FIG. 6 shows an alternative mesh generally indicated 600 comprising the random distribution of the first and second pluralities of apertures 610 and 620.

FIG. 7 shows an alternative embodiment with a mesh generally indicated 700 having a first plurality of apertures 710, a second plurality of apertures 720, and a third plurality of apertures 730. Where there are three pluralities of aperture, 710, 720, 730, the distribution and arrangements as shown in FIGS. 3 to 7 could also apply, and that the relative numbers of each aperture size can be varied. The three sizes of aperture could also be distributed randomly as shown in FIG. 6 or concentrically as shown in FIG. 5.

In the case of there being three sizes of aperture, the third plurality of apertures could be sized so as to produce droplets having a diameter of less than 1 μm. In use, droplets of less than 1 μm will not generally be absorbed by the body and will be exhaled, but will produce a cloud of vapour that could be part of the user experience. There may be further situations where a number of ingredients will want to be targeted to different parts of the respiratory tract and so four or more different aperture sizes may be provided.

Alternatively, the third plurality of apertures could be sized so that they produces droplets of 2-5 μm. Droplets of such a size will create a throat hit which may be part of the user experience.

In an alternative embodiment there may be four different average diameters of aperture arranged in any way as described herein. The four average aperture diameters could be size to produce droplets in the range of less than 1 μm, 1-5 μm, 5-10 μm, and greater than 10 μm respectively. Four different average aperture diameters could ensure that that some droplets will create a taste sensation, some droplets will cause a throat hit, some droplets will be absorbed by the alveoli and some droplets will be exhaled. This spectrum of droplet sizes would create the full range of user experience, such as that from smoking a cigarette.

In respect of the embodiments of mesh described herein the proportion of each average aperture diameter can be varied to optimise the desired user experience. In some aspects of the present invention a mesh nebuliser may be provided which may be an active or passive mesh nebuliser and in some embodiments the mesh may be interchangeable so that a user can experience different experiences whilst using a single nebulizing device.

A further arrangement of two sizes of aperture is shown in FIG. 10. There is shown a mesh generally indicated 1000 which provides a first plurality of apertures 1010 and a smaller second plurality of apertures 1020. The first plurality of apertures are arrange to extend radially from the centre of the mesh 1000 and the second plurality of apertures 1020 are arranged therebetween. Alternative arrangements could be provided whereby first, second, third or fourth or more pluralities of aperture are arranged radially or in a series of rows or crosshatches depending upon the user experience desired. The arrangement of aperture size could also be influenced based on performance, for example a particular arrangement of the smaller sizes of aperture could prevent them becoming clogged during use. With an active nebuliser the mesh will be generally domed shaped and will flex during use and so a particular arrangement may be beneficial for those embodiments.

According to a further aspect of the present invention there is shown in FIGS. 8 and 9 alternative apertures which have differently-sized regions, so that a single aperture can produce droplets of various sizes. There is shown in FIG. 8 aperture generally indicated 800 which is generally hourglass shaped and comprises a first region 810 and a second region 820 which has a smaller average diameter than the first region 810. The first and second regions 810 and 820 are conjoined by a narrow region 830.

As shown in FIG. 9, there is provided an alternative aperture generally indicated 900, which comprises three regions of different average diameter 910, 920 and 930 which are conjoined by narrow region 940. The apertures of FIGS. 8 and 9 may be produced using any known method of manufacture.

What is claimed is:

1. A mesh for use in a mesh nebuliser, the mesh comprising:
a first plurality of apertures extending through the mesh, the first plurality of apertures having a first average diameter; and
at least one second aperture extending through the mesh, the at least one second aperture having a second average diameter,
wherein the first average diameter is different to the second average diameter.

2. A mesh as claimed in claim 1, wherein the first plurality of apertures has an average diameter in the range of 0.5-2.5 μm and the at least one second aperture has a diameter in the range of 2.5-15 μm.

3. A mesh as claimed in claim 2, wherein the at least one second aperture has an average diameter in the range of 5-15 μm.

4. A mesh as claimed in claim 1, wherein the first plurality of apertures are sized so as to produce particle sizes with an MMAD in the range of 1-5 μm.

5. A mesh as claimed in claim 1, wherein the at least one second aperture is sized so as to produce particle sizes with an MMAD in the range of 5-30 μm.

6. A mesh as claimed in claim 5, wherein the at least one second aperture is sized so as to produce particle sizes with an MMAD in the range of 10-30 μm.

7. A mesh as claimed in claim 1, wherein the first plurality of apertures is arranged on one side of the mesh and the at least one second aperture is arranged on the other side of the mesh.

8. A mesh as claimed in claim 1, wherein the first plurality of apertures is arranged at the centre of the mesh and wherein the at least one second aperture is arranged on the outside of the first plurality of apertures.

9. A mesh as claimed in claim 1, wherein the first plurality of apertures and the at least one second aperture are distributed evenly on the mesh.

10. A mesh as claimed in claim 1, wherein the first plurality of apertures comprises a range of 0.1%-99% of the apertures by number and the at least one second aperture comprises a range of 0.1%-99% of the apertures by number, wherein the total proportion of both sizes of aperture amounts to 100%.

11. A mesh as claimed in claim 1, further comprising a third plurality of apertures, the apertures having an average diameter of less than 0.5 μm.

12. A mesh as claimed in claim 1, wherein the third plurality of apertures are sized so as to produce particle sizes with an MMAD of less than 1 μm.

13. A mesh nebuliser comprising a mesh as claimed in any of claim 1.

14. A mesh nebuliser as claimed in claim 13 wherein the mesh nebuliser is a passive nebuliser.

15. A mesh nebuliser as claimed in claim 13, wherein the mesh is interchangeable.
